(19) United States
(12) Patent Application Publication
(10) Pub. No.: US 2011/0218349 A1
(43) Pub. Date: Sep. 8, 2011

(54) Method for Producing Alkylene Oxide Addition Products

(75) Inventors: Stefan Franzen, Kamen (DE); Bernhard Gutsche, Hilden (DE); Bernd Fabry, Korschenbroich (DE); Eik Ulf Mahnke, Velbert (DE)

(73) Assignee: Cognis IP Management GmbH, Dusseldorf (DE)

(21) Appl. No.: 12/440,459

(22) PCT Filed: Aug. 29, 2007
(86) PCT No.: PCT/EP2007/007531

§ 371 Date: Mar. 7, 2009

(30) Foreign Application Priority Data
Sep. 7, 2006 (DE) 10 2006 041 903.0

(51) Int. Cl. C07D 301/10

(52) U.S. Cl. 549/534

(57) ABSTRACT

The invention relates to a method for producing alkylene oxide addition products. The method according to the invention is characterized by (a) contacting ethylene and/or propylene with an oxidizing agent in a first structured reactor (“μ reactor”) and (b) feeding the ethylene oxide and/or propylene oxide so obtained, optionally after purification, to a second structured reactor where it is reacted with a compound having a nucleophilic molecular group.

### FIELD OF THE INVENTION

The invention is in the field of preparation of nonionic surfactants and relates to a novel two-stage process for integrated preparation of alkylene oxide addition products in structured reactors.

### STATE OF THE ART

Alkylene oxides, such as ethylene oxide and propylene oxide, are some of the most important mineral oil-based industrial chemicals. Ethylene oxide (EO) in particular is a starting material for the preparation of ethylene glycol, which is added, for example, to aviation gasoline as an antifreeze. Since ethylene oxide and propylene oxide are additionally also depleted by all kinds of substances which possess acidic hydrogen atoms or have nucleophilic centers in any form, they are especially also suitable for addition onto alcohols or amines to form polyalkylene glycol chains which impart a hydrophilic character to these substances. The principal outlet for this type of compounds is nonionic surfactants, which find use especially in washing compositions and cosmetics.

Ethylene oxide and (to a minor degree) propylene oxide are prepared by direct oxidation of the corresponding alkenes over silver catalysts:

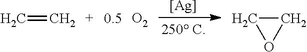

The reaction is, for example, exothermic at 120 kg/mol for ethylene oxide and competes with the complete combustion of the ethylene to carbon dioxide, which proceeds significantly more exothermically at more than 1300 kg/mol. Ethylene oxide is prepared industrially, for example, generally in tube bundle reactors which may contain up to 1000 individual tubes and are cooled from the outside by a liquid heat carrier, for example tetralin, in order to be able to maintain the oxidation temperature of from 230 to 270° C. even in the case of increasing total oxidation. The catalyst, for example 15% by weight of silver on Al₂O₃, is present as a bed in the tubes. In general, preference is given to oxidation with oxygen. Nevertheless, the conversion of ethylene is limited to from about 10 to 15%, since only in this way can selectivities of not more than 75 to 80% be achieved. About a quarter of the expensive starting material is thus combusted to carbon dioxide in this way. An additional factor is that typically up to 2.5% by volume of water and up to 10% by volume of carbon dioxide are present in the end product, and have to be removed with a high level of technical complexity before further utilization.

Before the reaction of ethylene oxide or propylene oxide, for example with alcohols, which leads to the formation of the technically important nonionic surfactant class of the alcohol polyglycol ethers, the gases have to be subjected to a complex purification. For this purpose, effective drying in particular is required, since water traces lead to the formation of polyethylene glycols, which are extremely undesirable as by-products. Subsequently, the carbon dioxide is bound in the form of potassium carbonate. The crude ethylene oxide is typically subjected to a three-stage distillation before it has the required purity, as has been required to date for the subsequent alkoxylation reaction.

The alkoxylation is typically performed batchwise, for example in stirred autoclaves or loop reactors at temperatures between 80 and 200° C.; alternatively, the liquid reaction mixture can also be dispersed into an alkylene oxide-containing gas phase. On this subject, reference may be made by way of example to a review article in Chem. Res. 25, 9482-9489 (2005). Typically, the compound with a nucleophilic center—for example an alcohol, a carboxylic acid, an ester or an amine—is initially charged together with the catalyst and then the desired amount of alkylene oxide is injected, which, depending on the temperature, generally establishes a pressure of up to 12 bar. Suitable catalysts are basic compounds, for example alkali metal alkoxides, or Lewis acids, the latter having the disadvantage that they tend to form considerable amounts of undesired polyglycol ethers.

Considering the overall process, the preparation of alkylene oxide addition products is associated with a whole series of technical and economic disadvantages:
in the course of ethylene oxide preparation, a large portion of the valuable feedstock is combusted to worthless carbon dioxide and water;
the workup of the crude ethylene oxide is technically extremely demanding, but necessary, since conventional alkoxylation reactors do not allow a lower purity and the presence of impurities;
the storage and especially the transport from the preparation of the alkylene oxide to the site where the alkoxylation takes place is problematic owing to the risk of explosion, and likewise associated with a high level of technical protection;
the existing alkoxylation processes allow only batchwise preparation of the alkylene oxide addition products.

The object of the present invention was thus to simultaneously solve the problems of the prior art mentioned by a comprehensive preparation process.

### DESCRIPTION OF THE INVENTION

The invention provides a process for preparing alkylene oxide addition products, wherein
(a) ethylene and/or propylene is contacted with an oxidizing agent in a first structured reactor (“μ-reactor”) and
(b) the ethylene oxide and/or propylene oxide obtained, optionally after purification, is fed into a second structured reactor in which it is reacted with a compound having a nucleophilic molecular group.

The proposed novel process offers the major economic advantage of obtaining the alkylene oxide at the same site at which the alkoxylation also takes place, such that complex and hazardous transport and/or storage are no longer required. The conversion of the alkylene oxides in the structured reactor makes the hitherto unavoidable distillative purification of the alkylene oxides superfluous. Furthermore, in the removal of the alkylene oxides from the alkene- and carbon dioxide-rich gas stream, complete purity of the ethylene oxide or propylene oxide need not be achieved either, since the impurities which remain in small concentrations do not disrupt the alkoxylation process, nor do they adversely affect the product quality. Instead, the low-boiling impurities from the EO/PO production are instead removed after the alkoxylation process in the course of the deodorization which is customary in any case. This has the further advantage that the boiling point differences between product of value and impurities (carbon dioxide, ethylene, formaldehyde, acetaldehyde) are significantly greater and the removal therefore becomes easier.

### Structured Reactors and Micro Reaction Systems

A central element of the present invention consists in the finding that structured reactors enable both the oxidation of ethylene and propylene and the subsequent alkoxylation to be performed irrespective of the explosion limits, since the reaction can be conducted isothermally, the reactants have only a minimal residence time in the reactor and the reaction channels have diameters which do not exceed the maximum experimental safe gap. The term “maximum experimental safe gap” is understood to mean the maximum diameter of a reactor at which a flame resulting from explosion is still automatically extinguished. These circumstances make it possible to use any mixtures of ethylene or propylene and oxidizing agent and nevertheless also to operate the reactor safely in the explosion range.

The term “structured reactor” is understood to mean an array of reaction channels which can be operated individually, in modules or else all together and are disposed in a matrix which serves for stabilization, securing, heating or cooling. A preferred embodiment of a structured reactor is that of micro reaction systems, which are also referred to in general as micro- or μ-reactors. They have the feature that at least one of the three dimensions of the reaction chamber has a measurement in the range from 1 to 2000 μm, and they thus feature a high transfer-specific inner surface area, short residence times of the reactants and high specific heat and mass transfer performances. A detailed article on this subject can be found, for example, in Jähnisch et al. in Angewandte Chemie Vol. 116, 410-451 (2004). Reference is made by way of example to European patent application EP 0903174 A1 (Bayer), in which the liquid phase oxidation of organic compounds in a microreactor consisting of an array of parallel reaction channels is described. Microreactors may additionally comprise microelectronic components as integral constituents. In contrast to known microanalytical systems, it is by no means necessary in the microreactors that all lateral dimensions of the reaction chamber are within the μm range. Instead, their dimensions are determined exclusively by the type of reaction. Accordingly, for particular reactions, useful microreactors are also those in which a particular number of microchannels is bundled, such that micro- and macrochannels or parallel operation of a multitude of microchannels may be present alongside one another. The channels are preferably arranged parallel to one another in order to enable a high throughput and to keep the pressure drop as low as possible.

Support

The supports (also referred to as “wafers”) in which the structure and dimensions of the micro reaction systems are defined may be material combinations, for example silicon-silicon, glass-glass, metal-metal, metal-plastic, plastic-plastic or ceramic-ceramic, or combinations of these materials, although the preferred embodiment is a silicon-glass composite, an aluminum oxide or a zeolite. Useful supports also include polyacrylates which are produced by layer-by-layer hardening and are particularly inexpensive to produce. A further alternative is that of HAT ceramics, specifically those which are surrounded by a pressure-resistant jacket, and also all-metal reactors in which the reaction channels are coated appropriately to prevent decomposition of the oxidizing agent. A support of thickness, for example, from 100 to 2000 μm, preferably about 400 μm, is structured preferably by means of suitable microstructuring or etching techniques, for example reactive ion etching, through which it is possible, for example, to manufacture three-dimensional structures irrespective of the crystal orientation in silicon [cf. James et al. in Sci. Am. 4, 248 (1993)]. It is also possible, for example, to treat microreactors of glass in the same way. Subsequently, catalysts customary for the oxidation or alkoxylation can then be applied to the supports by suitable microstructuring techniques, for example by saturation, impregnation, precipitation from the gas phase, etc.

Supports treated in this way may have from 10 to 1000, preferably from 100 to 500 and especially from 200 to 300 micro reaction systems running parallel to one another, which may be actuated and operated either in parallel or sequentially. The geometry, i.e. the two-dimensional profile of the channels, may be very different: possible profiles include straight lines, curves, angles and the like, and combinations of these shape elements. Not all micro reaction systems need have the same geometry. The structures feature measurements of from 50 to 1500 μm, preferably from 10 to 1000 μm, and vertical walls, the depth of the channels being from 20 to 1800 μm and preferably from about 200 to 500 μm. The cross sections of each micro reaction chamber, which may but need not be square, are generally in the order of magnitude of from 20×20 to 1500×1500 μm² and especially from 100×100 to 300×300 μm², as is specified as typical, for example, by Burns et al. in Trans IChemE 77(5), 206 (1999). To supply the micro reaction chambers with the reactants, the support is etched through at the points intended for this purpose.

Finally, the structured support is bonded by a suitable process, for example anodic bonding, to a further support, for example of glass, preferably Pyrex glass, and the individual flow channels are sealed tightly to one another. Of course, depending on the substrate material, other construction and bonding techniques are also possible to realize impervious flow systems, which will be apparent to the person skilled in the art, without any need for an inventive step for this purpose.

Structuring of the Microreactors

The micro reaction systems may be divided into one or more mixing zones, one or more reaction zones, one or more mixing and reaction zones, one or more heating and cooling zones, or any combinations thereof. The micro reaction systems preferably have three zones, specifically, as a result of which it is especially possible to efficiently perform two-stage or multistage reactions in the liquid phase or else the gaseous phase. In the first zone, the two reactants are mixed and reacted; in the second zone, the reaction between the product of the first zone and a further reactant takes place, while, in the third zone, the termination of the reaction is brought about by lowering the temperature. It is not absolutely necessary to thermally strictly separate the first reaction zone and the second reaction zone from one another. Specifically, when the addition of a further reactant is required or several mixing points are desired instead of one, this can also take place in reaction zone 2 over and above zone 1. The micro reaction systems may be operated sequentially or else simultaneously, i.e. in parallel with defined amounts of reactant in each case and have identical or different geometries. A further possible way in which the geometry of the micro reaction systems may differ consists in the mixing angle at which the reactants meet one another and which may be between 15 and 270° and preferably from 45 to 180°. Furthermore, it is possible to cool or to heat each of the three zones independently, or to vary the temperature within one zone as desired, the reaction chambers in this example being channels whose length per zone may be from 10 to 500 mm.

### Oxidation

The alkylene oxides, especially ethylene oxide and/or propylene oxide, can be prepared by direct oxidation of the corresponding alkenes. Useful oxidizing agents for this purpose include oxygen or air, but also peroxo compounds, for example hydrogen peroxide, and ozone. In this connection, reference is made in particular to an article by Schüth et al. in Ind. Eng. Chem. Res 41, 701-719 (2002), from which the use of microreactors for silver-catalyzed oxidation of ethylene to ethylene oxide is known, and whose content is part of the present patent application by reference. It has been found to be particularly effective, for oxidation of the alkenes, either to coat the micro reaction channels with silver—which can be deposited, for example, from the vapor phase—or to produce the structured reactor directly from this metal. Suitable cocatalysts or so-called promoters are halohydrocarbons, for example 1,2-dichloroethane; however, it is also possible to partially halogenate the silver. The thickness of the catalyst layer is preferably on average from 50 to 2000 nm and especially 100 to 1000 nm.

The oxidation reaction can be performed at temperatures in the range from 90 to 300° C., preferably from 120 to 280° C. and especially from 180 to 260° C., it being possible, depending on the oxidizing agent, to work either under reduced pressure (for example ozone) of 0.1 bar or at pressures of up to 30 bar (oxygen). A significant advantage is, as already mentioned at the outset, that the reaction can also be performed within the explosion limits of the mixtures of ethylene and/or propylene on the one hand, and oxidizing agent on the other hand, such that the mixing ratio of the feedstocks can be selected exclusively according to thermodynamic aspects and not with observation of safety guidelines. Equally, it has been found to be useful to add further inert gas, for example methane, to the mixtures of ethylene and/or propylene and oxidizing agent in a proportion of up to 70, preferably up to 60 and especially up to 50% by volume.

As already mentioned at the outset, the process according to the invention enables the use of ethylene oxide or propylene oxide in a significantly lower purity than has been required to date in the prior art processes. In particular, the complex distillation of the ethylene oxide can be dispensed with. In the context of the present invention, the workup is effected by drying the gas stream after it leaves the first micro reaction system and before it is fed into the second micro reaction system, in order to prevent the formation of undesired glycol in the subsequent alkoxylation. Subsequently, ethylene oxide and/or propylene oxide are condensed out of the gas stream after it leaves the first micro reaction system and then fed into the second micro reaction system in liquid form.

### Alkoxylation

The selection of the reactant for the subsequent alkoxylation is uncritical per se. The only condition is that it is a compound with a nucleophilic center, preferably with an acidic hydrogen atom. Useful for this purpose are especially alcohols of the formula (I)

R¹OH (I)

in which R¹ is a linear or branched hydrocarbon radical having from 1 to 22, preferably from 8 to 18, carbon atoms and from 0 or 1 to 3 double bonds. Typical examples are, in addition to the lower aliphatic alcohols methanol, ethanol and the isomeric butanols and pentanols, the fatty alcohols, specifically caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, and technical-grade mixtures thereof, which are obtained, for example, in the high-pressure hydrogenation of technical-grade methyl esters based on fats and oils, or aldehydes from the Roelen oxo process, and as a monomer fraction in the dimerization of unsaturated fatty alcohols. Preference is given to technical-grade fatty alcohols having from 12 to 18 carbon atoms, for example coconut fatty alcohol, palm fatty alcohol, palm kernel fatty alcohol or tallow fatty alcohol.

A further group of compounds which are suitable as starting materials for the alkoxylation is formed by the carboxylic acids of the formula (II)

R²CO—OH (II)

in which R²CO is a linear or branched acyl radical having from 1 to 22 carbon atoms and from 0 or 1 to 3 double bonds. Typical examples are in particular the fatty acids, specifically caproic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petrosilic acid, linoleic acid, linolenic acid, elaeostearic acid, arachic acid, gadoleic acid, behenic acid and erucic acid, and technical-grade mixtures thereof, which are obtained, for example, in the pressure cleavage of natural fats and oils, in the oxidation of aldehydes from the Roelen oxo process, or the dimerization of unsaturated fatty acids. Preference is given to technical-grade fatty acids having from 12 to 18 carbon atoms, for example coconut fatty acid, palm fatty acid, palm kernel fatty acid or tallow fatty acid. It will be appreciated that it is also possible to alkoxylate functionalized carboxylic acids, for example hydroxycarboxylic acids such as ricinoleic acid or citric acid, or dicarboxylic acids such as adipic acid and azelaic acid. Instead of the acids, it is also possible to use the corresponding esters with C₁-C₂₂ alcohols or glycerol; here, an insertion into the ester group then takes place.

Finally, a further group of compounds suitable as starting materials is also that of amines of the formula (III)

R³—NH—R⁴ (III)

in which R³ and R⁴ are each independently hydrogen, alkyl groups having from 1 to 18 carbon atoms or hydroxyalkyl groups having from 1 to 4 carbon atoms. Typical examples are methylamine, dimethylamine, ethylamine, diethylamine, methylethylamine, and the different propyl, butyl, pentyl and fatty amines of analogous structure.

The alkoxylation preferably takes place in the presence of catalysts which may be of homogeneous or heterogeneous nature. In the case of use of homogeneous catalysts, it is advisable to dissolve or to disperse them in the compounds with a nucleophilic center, i.e., for example, in an alcohol, and to supply them thus to the micro reaction system. Examples of suitable homogeneous catalysts include alkali metal hydroxides or alkali metal alkoxides, especially potassium hydroxide, potassium tert-butoxide and especially sodium methoxide. When heterogeneous catalysts are used, they are preferably used by coating the channels second micro reaction system. These layers then preferably have an average thickness of 50 to 2000 nm and especially from 100 to 1000 nm. A preferred example of a suitable catalyst, which is applied, for example, by impregnation and subsequent calcination, is hydrotalcite. The micro reactor for the alkoxylation is preferably a micro falling-film reactor, as described, for example, in publication DE 10036602 A1 (CPC); however, any other reactor type which enables contact between the phases in the thin layer is also suitable. In general, the ethylene oxide and/or propylene oxide and the compounds with a nucleophilic center are reacted in a molar ratio of from 1:1 to 200:1, preferably from 5:1 to 50:1 and especially from 8:1 to 20:1. The reaction temperature may vary between 50 and 200° C. depending on the starting material, and is preferably from 100 to 120° C., while the reaction can be carried out either under reduced pressure, for example 0.1 bar, or at pressures up to about 12 bar. The alkoxylation is generally followed by a deodorization. This reaction step can be utilized in order to remove impurities present in the ethylene oxide or propylene oxide, in order to provide a product which is on-spec from every point of view and corresponds to the existing prior art processes.

### EXAMPLES

### Example 1

Ethylene and air are heated to a temperature of 220° C. in a micro heat exchanger. This exploited the heat of the gas mixture emerging from the reactor. The two gas streams were contacted in a mixing unit and fed to the actual reactor. This part consisted of a plurality of silver foils stacked one on top of another, to which the actual catalyst had been applied. The reactor was operated at a pressure of 2 MPa. The residence time in the reactor was 0.5 second, within which full conversion of the oxygen present in the feed stream was achievable. The heat of reaction was removed by means of a pressurized water circuit. The reaction mixture left the reactor with a temperature of 250° C. and consisted of 73 mol % of the inert gas nitrogen, 3.7 mol % of unconverted ethylene, and 4.8 mol % of the product of value, ethylene oxide. From the parallel reactions, 16 mol % of carbon dioxide, 2 mol % of water and traces of formaldehyde and acetaldehyde were additionally present in the gas stream. The conversion was 61.7% at a selectivity of 79%.

The gas stream was cooled in two stages to a temperature of 90° C. and conducted through a fixed bed in which both the water and the aldehydes were bound adsorptively. The gas stream thus dried was decompressed to 1 MPa in several stages. This utilized the Joule-Thomson effect, in order to condense out the ethylene oxide. The condensate stream was subsequently collected in a reservoir vessel and contained 92.5 mol % of ethylene oxide, 7.2 mol % of carbon dioxide and small amounts of ethylene. The gas stream was sent to a workup, in which CO₂ was removed by acidic scrubbing, and then recycled into the reactor.

To prove the employability of the ethylene oxide thus obtained in the subsequent alkoxylation, a technical-grade C_{12/14} coconut fatty alcohol mixture (Lorol® Spezial, Cognis Deutschland GmbH & Co. KG) was preheated to 130° C. in a flow heater. A 45% by weight aqueous potassium hydroxide solution was then metered into the raw material stream, so as to establish a KOH concentration of approx. 0.1% by weight. The mixture thus obtained was freed of the water in a continuous micro falling-film reactor at a temperature of approx. 130° C. and a reduced pressure of approx. 200 mbar.

Both the dried feed stream and the ethylene oxide from the collecting vessel of the upstream oxidation process were compressed to a pressure of 30 bar with the aid of two pumps and metered together via a liquid distributor into a micro tube bundle reactor consisting of 50 stainless steel capillaries with a length of 25 cm and a diameter of 1 mm. The heat released in the addition of the ethylene oxide onto the alcohol led to a rise in the temperature in the reactor to from 165 to 180° C. For this reason, the tube bundle reactor was cooled with a pressurized water circuit, with the aid of which it was ensured that the alkoxylation product leaving the tube bundle had only a temperature of 80° C. The heat removed by means of the cooling water circuit was utilized in order to preheat further fatty alcohol to the temperature of 130° C. After the alkoxylation, the product mixture was decompressed to 0.15 MPa and carbon dioxide residues which were present as a result were removed. The subsequent analysis of the product showed that the presence of the carbon dioxide had no adverse effect on the product quality. Instead, the presence of the carbon dioxide led to inertization and hence to an increase in the safety of the alkoxylation process studied.

### Example 2

Ethylene, and oxygen admixed with methane and argon, are heated to a temperature of 220° C. in a micro heat exchanger. This exploited the heat of the gas mixture emerging from the reactor. The two gas streams were contacted in a mixing unit and fed to the actual reactor. This part consisted of a plurality of silver foils stacked one on top of another, to which the actual catalyst had been applied. The reactor was operated at a pressure of 2 MPa. The residence time in the reactor was 0.5 second, within which full conversion of the oxygen present in the feed stream was achievable. The heat of reaction was removed by means of a pressurized water circuit. The reaction mixture left the reactor with a temperature of 250° C. and consisted of 40.2 mol % of the inert gases argon and methane, 35.3 mol % of unconverted ethylene, and 5.0 mol % of the product of value, ethylene oxide. From the parallel reactions, 18.1 mol % of carbon dioxide, 1.5 mol % of water and traces of formaldehyde and acetaldehyde were additionally present in the gas stream. The conversion was 14.2% at a selectivity of 86.8%.

The gas stream was cooled in two stages to a temperature of 90° C. and conducted through a fixed bed in which both the water and the aldehydes were bound adsorptively. The gas stream thus dried was decompressed to 1 MPa in several stages. This utilized the Joule-Thomson effect, in order to condense out the ethylene oxide. The condensate stream was subsequently collected in a reservoir vessel and contained 92.5 mol % of ethylene oxide, 7.2 mol % of carbon dioxide and small amounts of ethylene. The gas stream was sent to a workup, in which CO₂ was removed by acidic scrubbing, and then recycled into the reactor.

To prove the employability of the ethylene oxide thus obtained in the subsequent alkoxylation, a technical-grade C_{12/14} coconut fatty alcohol mixture (Lorol® Spezial, Cognis Deutschland GmbH & Co. KG) was preheated to 130° C. in a flow heater. A 45% by weight aqueous potassium hydroxide solution was then metered into the raw material stream, so as to establish a KOH concentration of approx. 0.1% by weight. The mixture thus obtained was freed of the water in a continuous micro falling-film reactor at a temperature of approx. 130° C. and a reduced pressure of approx. 200 mbar. Both the dried feed stream and the ethylene oxide from the collecting vessel of the upstream oxidation process were compressed to a pressure of 30 bar with the aid of two pumps and metered together via a liquid distributor into a micro tube bundle reactor consisting of 50 stainless steel capillaries with a length of 25 cm and a diameter of 1 mm. The heat released in the addition of the ethylene oxide onto the alcohol led to a rise in the temperature in the reactor to from 165 to 180° C. For this reason, the tube bundle reactor was cooled with a pressurized water circuit, with the aid of which it was ensured that the alkoxylation product leaving the tube bundle had only a temperature of 80° C. The heat removed by means of the cooling water circuit was utilized in order to preheat further fatty alcohol to the temperature of 130° C. After the alkoxylation, the product mixture was decompressed to 0.15 MPa and carbon dioxide residues which were present as a result were removed. The subsequent analysis of the product showed that the presence of the carbon dioxide had no adverse effect on the product quality. Instead, the presence of the carbon dioxide led to inertization and hence to an increase in the safety of the alkoxylation process studied.

1. A process for preparing alkylene oxide addition products, characterized in that
(a) ethylene and/or propylene is contacted with an oxidizing agent in a first structured reactor (“μ-reactor”) and
(b) the ethylene oxide and/or propylene oxide obtained, optionally after purification, is fed into a second structured reactor in which it is reacted with a compound having a nucleophilic molecular group.

2. The process as claimed in claim 1, characterized in that the structured reactors are micro reaction systems.

3. The process as claimed in claim 2 and/or 3, characterized in that the micro reaction systems have been applied to supports.

4. The process as claimed in at least one of claims 1 to 3, characterized in that the micro reaction systems have at least one inlet for the reactants and at least one outlet for the products.

5. The process as claimed in at least one of claims 1 to 4, characterized in that the support is a silicon-glass composite, an alumina or a zeolite.

6. The process as claimed in at least one of claims 1 to 5, characterized in that catalysts customary for the oxidation or alkoxylation are applied to the support by suitable microstructuring techniques.

7. The process as claimed in at least one of claims 1 to 6, characterized in that each support has 10 to 1000 micro reaction systems running parallel to one another, which can be accessed sequentially or simultaneously by the reactants.

8. The process as claimed in at least one of claims 1 to 7, characterized in that the micro reaction systems all have the same geometry or different geometries.

9. The process as claimed in at least one of claims 1 to 8, characterized in that the micro reaction systems have, in at least one dimension, measurements in the range from 20 to 1500 μm.

10. The process as claimed in at least one of claims 1 to 9, characterized in that the micro reaction systems have a depth of 20 to 1800 μm.

11. The process as claimed in at least one of claims 1 to 10, characterized in that the micro reaction systems have cross sections of from 20×20 to 1500×1500 μm².

12. The process as claimed in at least one of claims 1 to 11, characterized in that the micro reaction systems are channels which have a length of 1 to 1000 mm.

13. The process as claimed in at least one of claims 1 to 12, characterized in that the micro reaction systems have one or more mixing zones, one or more reaction zones, one or more mixing and reaction zones, one or more heating or cooling zones or any combinations thereof.

14. The process as claimed in at least one of claims 1 to 13, characterized in that the channels in the first micro reaction system have been coated with silver and optionally further cocatalysts (“promoters”).

15. The process as claimed in claim 14, characterized in that the thickness of the catalyst layer is on average 50 to 2000 nm.

16. The process as claimed in at least one of claims 1 to 15, characterized in that the oxidizing agent used is oxygen and/or peroxo compounds.

17. The process as claimed in at least one of claims 1 to 16, characterized in that the oxidation is performed at temperatures in the range from 90 to 300° C.

18. The process as claimed in at least one of claims 1 to 17, characterized in that the reaction is performed within the range from 0.1 to 30 bar.

19. The process as claimed in at least one of claims 1 to 18, characterized in that the reaction is performed within the explosion limits of the mixtures of ethylene and/or propylene on the one hand, and oxidizing agent on the other hand.

20. The process as claimed in at least one of claims 1 to 19, characterized in that further inert gas is added to the mixtures of ethylene and/or propylene and oxidizing agent.

21. The process as claimed in at least one of claims 1 to 20, characterized in that the gas stream is dried after it leaves the first micro reaction system and before it is fed into the second micro reaction system.

22. The process as claimed in at least one of claims 1 to 21, characterized in that the ethylene oxide and/or propylene oxide is condensed out of the gas stream after it leaves the first micro reaction system and then fed into the second micro reaction system in liquid form.

23. The process as claimed in at least one of claims 1 to 22, characterized in that the compounds with a nucleophilic center used are alcohols of the formula (I)
R¹OH (I)
in which R¹ is a linear or branched hydrocarbon radical having from 1 to 22 carbon atoms and from 0 or 1 to 3 double bonds.

24. The process as claimed in at least one of claims 1 to 22, characterized in that the compounds with a nucleophilic center used are carboxylic acids of the formula (II)
R²CO—OH (II)
in which R²CO is a linear or branched acyl radical having from 1 to 22 carbon atoms and from 0 or 1 to 3 double bonds.

25. The process as claimed in at least one of claims 1 to 22, characterized in that the compounds with a nucleophilic center used are amines of the formula (III)
R³—NH—R⁴ (III)
in which R³ and R⁴ are each independently hydrogen, alkyl groups having from 1 to 18 carbon atoms or hydroxyalkyl groups having from 1 to 4 carbon atoms.

26. The process as claimed in at least one of claims 1 to 25, characterized in that the alkoxylation is performed in the presence of homogeneous or heterogeneous catalysts.

27. The process as claimed in claim 26, characterized in that the homogeneous catalysts are dissolved or dispersed in the compounds with a nucleophilic center.

28. The process as claimed in claims 26 and 27, characterized in that the homogeneous catalysts used are alkali metal hydroxides or alkali metal alkoxides.

29. The process as claimed in claim 28, characterized in that the channels of the second micro reaction system are coated with the heterogeneous alkoxylation catalysts.

30. The process as claimed in claim 29, characterized in that the layer has an average thickness of 50 to 2000 nm.

31. The process as claimed in claims 29 and/or 30, characterized in that the heterogeneous catalysts used are hydrotalcites.

32. The process as claimed in at least one of claims 1 to 31, characterized in that the alkoxylation is carried out in a micro falling-film reactor.

33. The process as claimed in at least one of claims 1 to 32, characterized in that the ethylene oxide and/or propylene oxide and the compound with a nucleophilic center are reacted in a molar ratio of from 1:1 to 200:1.

34. The process as claimed in at least one of claims 1 to 33, characterized in that the alkoxylation is performed at temperatures in the range from 50 to 200° C.

35. The process as claimed in at least one of claims 1 to 34, characterized in that the alkoxylation is performed at pressures of from 0.1 to 12 bar.
